(19) United States
(12) Patent Application Publication
(10) Pub. No.: US 2012/0208290 A1
(43) Pub. Date: Aug. 16, 2012

(54) METHOD FOR ENHANCING MASS OF GOLD NANOPARTICLE THROUGH LIGHT-IRRADIATION, METHOD AND SENSOR FOR DETECTING MOLECULAR BINDING USING THE METHOD FOR ENHANCING MASS

(75) Inventors: Soo Suk LEE, Suwon-si (KR); Hye Jung SEO, Chuncheon-si (KR); Sang Min JEON, Pohang-si (KR); Youn Suk CHOI, Yongin-si (KR); Hun Joo LEE, Hwaseong-si (KR); Jung Nam LEE, Incheon (KR); Joon Hyung LEE, Yongin-si (KR); Kyung Yeon HAN, Seoul (KR); Sang Kyu KIM, Yongin-si (KR); Yeol Ho LEE, Seoul (KR); Jae Phil DO, Seoul (KR)

(73) Assignees: POSTECH ACADEMY INDUSTRY FOUNDATION, Pohang-si (KR); SAMSUNG ELECTRONICS CO., LTD., Suwon-si (KR)

(21) Appl. No.: 13/215,822

(22) Filed: Aug. 23, 2011

(30) Foreign Application Priority Data
Feb. 16, 2011 (KR) 10-2011-0013641

(51) Int. Cl. G01N 33/84; B01J 19/12; G01N 30/00; B82Y 15/00

(52) U.S. Cl. 436/501; 422/69; 204/157.21; 977/773; 977/810; 977/902

(57) ABSTRACT

Provided are a sensor for detecting molecular binding by increasing the mass of a gold nanoparticle through light-irradiation and a method thereof. In the method, light-irradiation increases the size of gold nanoparticles without using a reducing agent, to enhance the mass. Accordingly, selectivity may be improved, and the sensitivity of detection may be improved due to a change in various properties of a gold nanoparticle.

### CROSS-REFERENCE TO RELATED APPLICATION

This application claims priority to Korean Patent Application No. 10-2011-0013641, filed on Feb. 16, 2011, and all the benefits accruing therefrom under 35 U.S.C. §119, the content of which in its entirety is herein incorporated by reference.

### BACKGROUND

1. Field

The disclosure relates to a sensor, which detects molecular binding with high-sensitivity by enhancing the mass of gold nanoparticles through light-irradiation, and a method thereof.

2. Description of the Related Art

A sensor such as a biosensor typically causes a change in an electrical or optical signal using, for example, a specific binding, reaction, etc. between a biomolecule, such as a protein, deoxyribonucleic acid (DNA), virus, bacteria, cell, and tissue, and a surface of the biosensor, thereby quantitatively and qualitatively analyzing and diagnosing the biomolecule.

A method of increasing the size of a gold nanoparticle using gold ions or silver ions together with a chemical reducing agent on the gold nanoparticle to enhance the mass of the gold nanoparticle, which is referred to as staining, is known. Since the method of enhancing the mass of a gold nanoparticle can cause binding of the gold nanoparticle after an antigen-antibody reaction of biomolecules and amplify a signal by enhancing gold or silver, complex preprocessing is not required. However, staining requires a catalyst, such as hydroxylamine or hydroquinone, to enhance the mass of the gold nanoparticle. Thus, gold or silver may be extracted from a solution as well as the gold nanoparticle, which deteriorates selectivity and sensitivity.

### SUMMARY

A method of enhancing the mass of a gold nanoparticle by increasing the size of a gold nanoparticle without using a reducing agent is disclosed.

Also, a method of detecting molecular binding and a sensor capable of improved sensitivity through a change in a mass, optical property, and/or electrical property of a gold nanoparticle is disclosed. The method may be used to detect biomolecular binding.

According to an aspect, a method of enhancing mass of a gold nanoparticle through light irradiation, including irradiating a composition comprising a gold nanoparticle and a metal-enhancing component with a wavelength of light effective to reduce the metal-enhancing component on a surface of the gold nanoparticle to increase the mass of the gold nanoparticle, is provided.

In the method, the light may be ultraviolet (“UV”) light.

In the method, the metal-enhancing component may be metal ions.

In this case, the metal ion may be selected from silver (Ag) ions, copper (Cu) ions, gold (Au) ions, and palladium (Pd) ions.

In the method, the gold nanoparticle may have a diameter of about 5 nm to about 200 nm.

According to another aspect, disclosed is a method of detecting molecular binding, including binding a target molecule to a sensor for detecting a change in a property of a gold nanoparticlebound to the target molecule; binding a gold nanoparticle to the target molecule; contacting the sensor including the bound gold nanoparticle and bound target moleculewith a composition including a metal-enhancing component; irradiating the composition and the bound gold nanoparticle with a wavelength of light effective to reduce the metal-enhancing component on a surface of the gold nanoparticle to change the property of the gold nanoparticle; and detecting the change in the property of the gold nanoparticle to detect the molecular binding of the target molecule.

In the method, the change in the properties of the gold nanoparticle may be selected from a change in mass, optical property, and electrical property.

In the method, the light may be UV light.

In the method, the metal-enhancing component may be a metal ion selected from a silver (Ag) ion, copper (Cu) ion, gold (Au) ion, and palladium (Pd) ion.

In the method, the gold nanoparticle may have a diameter of about 5 nanometers (nm) to about 200 nm.

In the method, the sensor may be a biosensor and the target molecule may be a biomolecule.

Thus, also disclosed is a method of detecting molecular binding of a target antigen or a target antibody to a sensor. The method includes binding the target antigen or target antibody to a surface of a sensor for detecting a change in a property of a gold nanoparticle bound to the antigen or antibody; binding a gold nanoparticle to the antigen or antibody bound to the sensor; contacting the sensor with the bound gold nanoparticle and bound target antigen or target antibody with a composition comprising a metal-enhancing component; irradiating the contacted composition with a wavelength of light effective to reduce the metal-enhancing component on a surface of the gold nanoparticle to change the property of the gold nanoparticle, wherein reducing is in the absence of a reducing agent; and detecting the change in the property of the gold nanoparticle to detect the molecular binding of the target antigen or target antibody to the sensor.

The target antigen or antibody may be directly or indirectly bound to the sensor and/or the gold nanop article.

According to another aspect, a sensor including a sensor, a gold nanoparticle, a composition, and a light irradiation device is provided. A target molecule binds to a surface of the sensor, and the sensor detects a change in a property of a gold nanoparticle bound to the target molecule. The gold nanoparticle is bound to the target molecule on a surface of the sensor. The composition includes a metal-enhancing component which changes the property of the gold nanoparticle, and the sensor is contacted with the composition. The light irradiation device irradiates the composition in contact with the sensor and the gold nanoparticle-bound target molecule. In the sensor, the metal-enhancing component is reduced on a surface of the gold nanoparticle by light from the light irradiation device to change the property of the gold nanoparticle, and the sensor detects the change in the property of the gold nanoparticle.

The sensor may be a biosensor and the target molecule may be a biomolecule.

In the sensor, the sensor may be selected from a mass sensor, an optical sensor, and an electrical sensor.

In the sensor, the change in the property of the gold nanoparticle may be selected from a change in mass, optical property, and electrical property.

In the sensor, the light may be UV light.

In the sensor, the metal-enhancing component may be metal ions, and the metal ions may be selected from silver (Ag) ions, copper (Cu) ions, gold (Au) ions, and palladium (Pd) ions.

In the sensor, the gold nanoparticle may have a diameter of about 5 nm to about 200 nm.

According to an embodiment, the mass of a gold nanoparticle may be enhanced by increasing the size of the gold nanoparticle without using a reducing agent, and thus selectivity or sensitivity may be improved.

Also, a gold-nanoparticle-based metal enhancement reaction by light-irradiation not only increases the mass and size of the gold nanoparticle but also changes optical and electrical properties, and thus it may be applied to a variety of sensors.

Further, sensitivity of detection may be improved due to a change in various properties of a gold nanoparticle due to the metal-enhancement reaction by light irradiation.

### BRIEF DESCRIPTION OF THE DRAWINGS

The above and other aspects, advantages and features of this disclosure will become more apparent by describing in further detail embodiments thereof with reference to the accompanying drawings, in which:

FIG. 1 illustrates an embodiment of a process that selectively causes a metal enhancement reaction on surfaces of gold nanoparticles through light-irradiation;

FIG. 2 is a flowchart illustrating an embodiment of a method of detecting molecular binding through light-irradiation;

FIG. 3 is a graph showing absorbance (optical density, O.D.) versus wavelength (nanometers, nm) of an embodiment of a gold nanoparticle in the presence of a metal-enhancing component before and after light-irradiation;

FIG. 4 is a photograph showing a change in color of an embodiment of a gold nanoparticle in the presence of a metal-enhancing component before and after light-irradiation;

FIG. 5 is a transmission electron microscope (TEM) image of an embodiment of a gold nanoparticle whose size has increased due to light-irradiation; and

FIG. 6 is a graph showing a frequency shift −Δf (hertz, Hz) versus metal-enhancing component concentration (millimolarity, mM) for data acquired by a quartz crystal microbalance of an embodiment of a gold nanoparticle subjected to a mass-increasing reaction.

### DETAILED DESCRIPTION

The invention now will be described more fully hereinafter with reference to the accompanying drawings, in which a non-limiting embodiment is shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including,” when used in this specification, specify the presence of stated regions, integers, steps, operations, elements, and/or components, but do not preclude the presence or addition of one or more other regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the disclosure, and will not be interpreted in an idealized or overly formal sense unless expressly so defined herein.

One or more embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear portions. Moreover, sharp angles that are illustrated may be rounded. Thus, the regions illustrated in the figures are schematic in nature and their shapes are not intended to illustrate the precise shape of a region and are not intended to limit the scope of the claims.

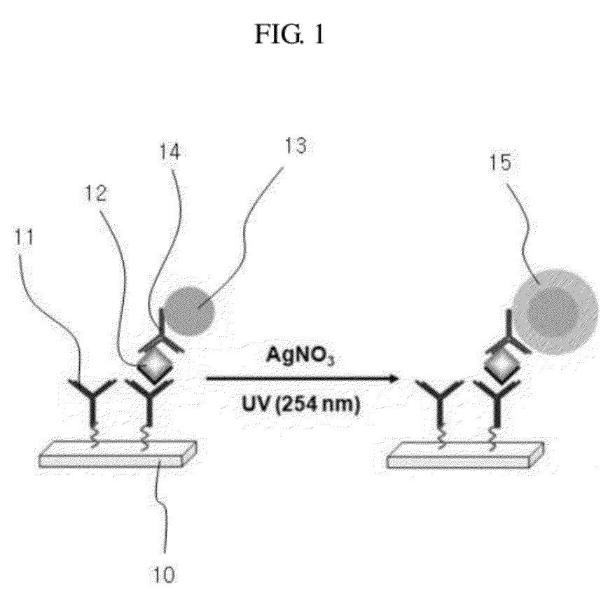

FIG. 1 illustrates an embodiment of a process that selectively causes a metal enhancement reaction on surfaces of a gold nanoparticle by light irradiation.

Referring to FIG. 1, a first antibody 11 fixed on the surface of a sensor 10 reacts to an antigen 12, and a second antibody 14 to which a gold nanoparticle 13 is bound reacts thereto. A source of silver, for example, silver nitrate (AgNO₃) is added to this system, and the system is irradiated with ultraviolet (“UV”) light having a wavelength of about 254 nanometers (“nm”). Then, a silver component 15 of the silver nitrate is reduced onto a surface of the gold nanoparticle 13, to increase the mass of the gold nanoparticle.

The gold nanoparticle-based metal enhancement reaction by light-irradiation not only increases the mass and size of the gold nanoparticle 13 but may also change one or more optical and/or electrical properties of the gold nanoparticle 13. Thus, the gold-nanoparticle-based metal enhancement reaction may be utilized in a variety of sensors. That is, the sensor 10 may be any one of a mass-based sensor, an optical sensor, and an electrical sensor. For example, as the mass-based sensor, a quartz crystal microbalance (“QCM”), a cantilever sensor, a surface acoustic wave (“SAW”) sensor, and the like may be used. As the optical sensor, a sensor using UV-visible spectrophotometry, colorimetry, surface plasmon resonance (“SPR”), and the like may be used. As the electrical sensor, an electrochemical sensor, a field effect transistor (“FET”) sensor, and the like may be used.

FIG. 1 is merely an embodiment, and the sensor is not limited to the embodiment. For example, as has been described with respect to the embodiment, UV light having a wavelength of about 254 nm is irradiated, but the wavelength is not limited thereto. In an embodiment, the wavelength of the light is that wavelength effective to reduce the metal-enhancing component to increase the mass of the gold nanoparticle 13. Such wavelengths will vary depending on the metal-enhancing component used and the sensor environment, and thus may be any effective wavelength, for example about 1 nm to about 1 millimeter (“mm”). In an embodiment, UV light have a wavelength of about 10 nm to about 380 nm is used. Also, it has been described that the silver component 15 from the silver nitrate is reduced on the surface of the gold nanoparticle 13 to increase the mass of the gold nanoparticle 13, but metal ions such as copper (Cu) ions, gold (Au) ions, and palladium (Pd) ions may also be used as a metal-enhancing component rather than silver (Ag) ions. A combination of different ions can be used. The metal-enhancing component may be supplied to the gold nanoparticle in any form that is reducible on a surface of the gold nanoparticle 13 upon irradiation. The metal-enhancing component is conveniently supplied to the system in the form of a salt, acid, or base of the metal. The type of salt used will depend on factors such as the metal-enhancing component, the environment of the sensor (e.g., whether aqueous or mixed organic-aqueous), the cost and commercial availability of the salt, and inertness of the counterion. For example, when silver is used as the metal-enhancing component, the silver 15 may be derived from a silver-containing compound such as a silver salt, for example, AgF, AgCl, AgBr, AgClO₄, Ag₂SO₄, Ag₂C₂H₃O₂, Ag₂C₂O₄, AgClO₃, AgCNO, silver triflate (AgOS(O₂)CF₃). Other forms where the silver is weakly bound to another moiety, can be used although the source of silver is not limited thereto. In an embodiment, the gold nanoparticles 13 may have an average diameter of about 5 nm to about 200 nm, or about 5 nm to about 175 nm, or about 5 nm to about 150 nm.

The gold nanoparticle-based metal enhancement reaction by light irradiation illustrated in FIG. 1 may be used in a method of detecting molecular binding, in particular biomolecular binding. In an embodiment, the reaction may be used to detect, for example, a synthetically produced compound that may interact with first antibody 11 and the second antibody 14 with gold nanoparticle 13. In another embodiment, a biomimetic epitope may be used to bind to the first and second antibodies 11 and 14.

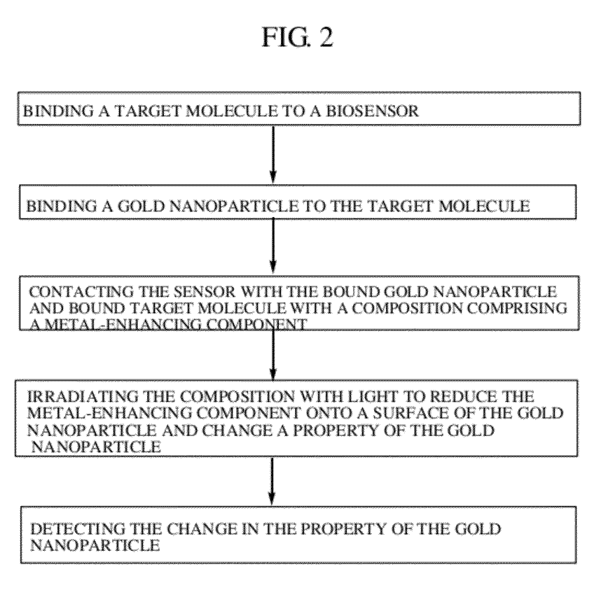

FIG. 2 is a flowchart illustrating an embodiment of a method of detecting molecular binding by light-irradiation.

Referring to FIG. 2, a sensor for detecting a change in a property of a gold nanoparticle binds to the target molecule. The target molecule is one that is capable of binding to the sensor and to a gold nanoparticle. In an embodiment, the target molecule is a biomolecule. The biomolecule may be an antigen, antibody, deoxyribonucleic acid (“DNA”), and ribonucleic acid (“RNA”). As used herein, DNA and RNA includes oligomers and polymers. In another embodiment, the biomolecule may be a lipid, vitamin, hormone, neurotransmitter, metabolite, peptide, oligosaccharide, or the like. Subsequently, a gold nanoparticle is bound to the target molecule. The gold nanoparticle may be specifically bound to the target molecule. The gold nanoparticle may be directly bound to the target molecule or indirectly bound to the target molecule via a probe to which the target molecule is specifically bound. For example, the gold nanoparticle may be attached to an antibody that can bind to an antigen bound to a sensor surface, wherein the antigen is also directly bound to the target antibody by a specific binding reaction. As another example, the gold nanoparticle may be indirectly bound to the probe by a reaction in which a first antibody is attached to the sensor surface and the gold nanoparticle is attached to a second antibody specifically binding to a target antigen. Alternatively, an antibody bearing the gold nanoparticle can be bound to an antigen bound to the sensor surface, and also to a target antigen on another binding domain of the antibody.

Next, the sensor with the gold nanoparticle-bound target molecule is contacted, for example immersed, in a composition including a metal-enhancing component.

Next, the composition is irradiated with light. Upon irradiation of the composition, the metal-enhancing component is reduced on a surface of the gold nanoparticle. As a result, a change in a property of the gold nanoparticle occurs.

Thereafter, the change in the property of the gold nanoparticle is detected.

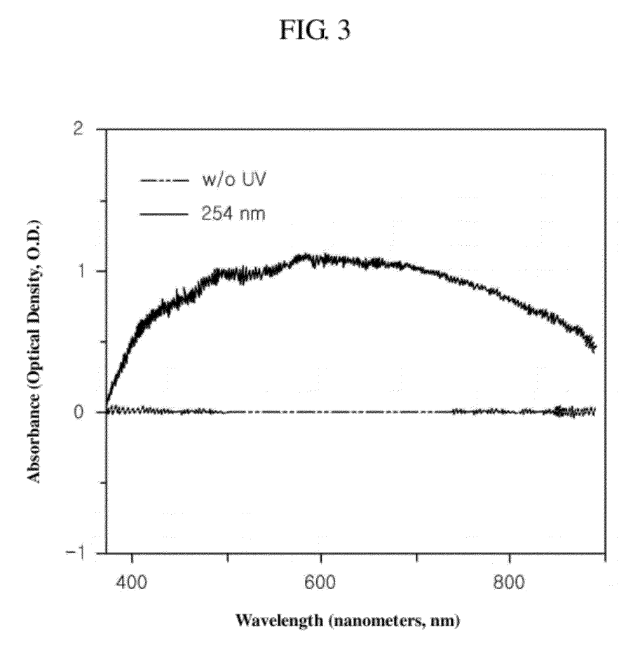

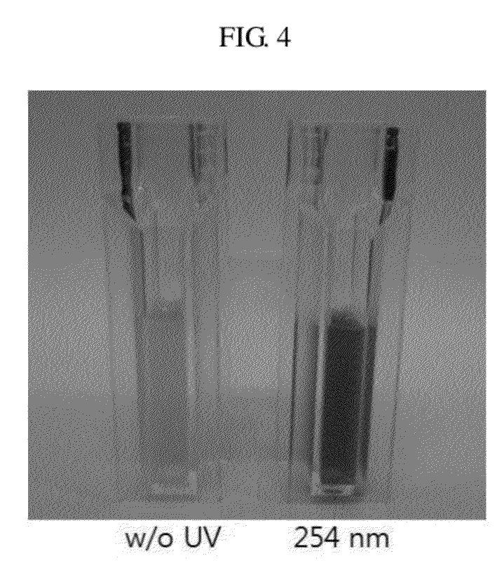

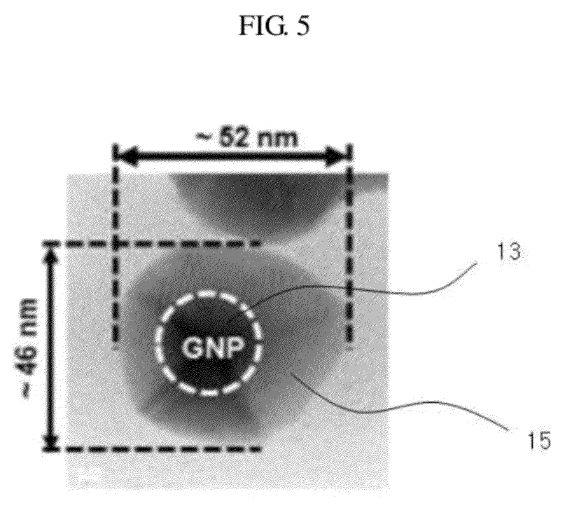

FIGS. 3 to 5 show a change in properties of an embodiment of a gold nanoparticle in the presence of a metal-enhancing component before and after light-irradiation. FIG. 3 is a graph showing a change in absorbance (optical density, O.D.) versus wavelength (nanometers, nm) of an embodiment of a gold nanoparticle in the presence of a metal-enhancing component before and after light-irradiation. FIG. 4 is a photograph showing a change in color of an embodiment of a gold nanoparticle in the presence of a metal-enhancing component before and after light-irradiation, and FIG. 5 is a transmission electron microscope (“TEM”) image of an embodiment of a gold nanoparticle whose size has increased due to light-irradiation.

To confirm a change in a property of the gold nanoparticle, a composition obtained by combining gold nanoparticles having an average size of about 20 nm and a silver nitrate (or in another embodiment chloroauric acid (HAuCl₄)) solution at a volume ratio of about 1:9 of gold nanoparticles to metal source is irradiated with UV light having a wavelength of about 254 nm for 10 minutes to reduce silver (or gold) ions in the composition on the surfaces of the gold nanoparticles.

Without being bound by theory, it is believed that the reduction of the metal-enhancing component, for example, silver ion, causes the metal (e.g., silver) to physisorb or chemisorb on the gold nanoparticles. As the metal is adsorbed, the surface sites of the gold nanoparticle may saturate so that additional reduced metal forms a metal adlayer on the adsorbed metal that is adsorbed on the gold nanoparticle.

Subsequently, absorbance is measured by UV-visible spectrophotometry. As a result of modification of the gold nanoparticle by the metal-enhancing component, absorbance is significantly varied as shown in FIG. 3. For example, the absorption spectrum appearing as a solid curve in FIG. 3 corresponds to the gold nanoparticle modified with the metal-enhancing component due to irradiation with UV light of the composition containing the gold nanoparticle and surface enhancing component. Similarly, the absorption spectrum appearing as a dashed curve in FIG. 3 corresponds to the nascent composition that has not been irradiated with UV light. Also, a change in color is observed by colorimetry so that the color before and after light-irradiation may be determined as shown in FIG. 4. Here, the liquid on the left does not absorb much visible light, while the 254 nm-irradiated sample shown on the right side of FIG. 4 exhibits noticeable darkening due to perceptible absorption of visible wavelengths that may be associated with the upper absorption spectrum appearing in FIG. 3 having a broad absorption peak about 580 nm.

In an embodiment, the breadth of the absorption peak in the visible wavelength range that corresponds to modification of the gold nanoparticle by the metal-enhancing component (e.g., silver or gold) may be varied by controlling the amount of the metal-enhancing component that adsorbs onto the gold nanoparticle. In another embodiment, the breadth of the peak may be controlled by an identity of the metal-enhancing component. In a further embodiment, the peak wavelength of the absorption spectrum may be tuned by controlling the amount of the metal-enhancing component adsorbed onto the gold nanoparticle. In yet another embodiment, the peak wavelength of the absorption spectrum may be tuned by controlling the identity of the metal-enhancing component adsorbed onto the gold nanoparticle. In yet a further embodiment, a hybrid component may be formed on the gold nanoparticle from reducing a metal-enhancing component containing multiple types of metal ions, for example, gold and silver ions.

Referring to FIG. 5, a micrograph acquired by a transmission electron microscope shows that a gold nanoparticle (“GNP”) having a size of about 20 nm increases to a size of about 46 nm to about 52 nm, that is, about twice or more of its original size, due to mass enhancement initiated by 255 nm irradiation.

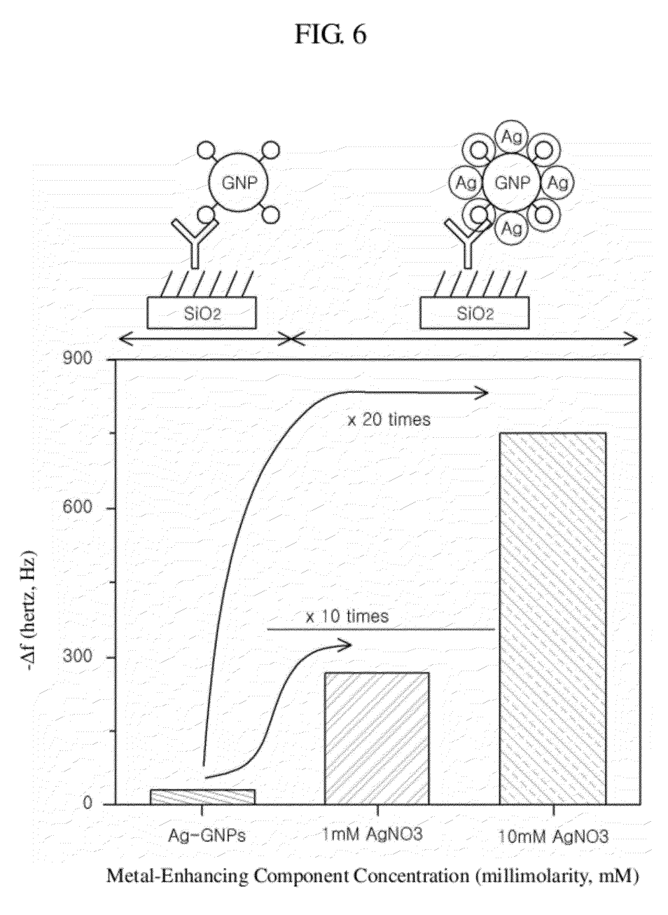

FIG. 6 is a graph showing frequency shift −Δf (hertz, Hz) versus metal-enhancing component concentration for data acquired by a quartz crystal microbalance of an embodiment of a gold nanoparticle in the presence of a metal-enhancing component.

Referring to FIG. 6, after zearalenone (a toxin) is bound on the surface of a gold nanoparticle having a size of about 20 nm, an antibody is bound on a surface of a QCM, which is coated with SiO₂, and the degree of binding between the gold nanoparticle to which the zearalenone is attached and the antibody on the surface of the QCM is measured using the QCM. After binding the zearalenone fixed on the gold nanoparticle to the antibody, a 1 millimolar (mM) or 10 mM silver nitrate solution is added to the sample, and UV light having a wavelength of about 254 nm is subsequently irradiated to the composition for 10 minutes, to reduce silver ions on the surface of the gold nanoparticle. After the mass enhancement by the silver ion reduction reaction, mass-sensitivity of the QCM to the zearalenone bound to the antibody improves about 10 to about 20 times according to a concentration of the silver nitrate. The results shown in FIG. 6 indicate that a mass-increasing reaction involving a gold nanoparticle and a metal-enhancing component, e.g., Ag⁺ from silver nitrate, enhances the sensitivity of a QCM as a detector for a biomolecule, for example, zearalenone.

While the invention has been particularly shown and described with reference to embodiments thereof, it will be understood by those of ordinary skill in the art that various changes in form and details may be made therein without departing from the spirit or scope of the present invention as defined by the following claims.

What is claimed is:

1. A method of enhancing mass, comprising:
irradiating a composition comprising a gold nanoparticle and a metal-enhancing component with a wavelength of light effective to reduce the metal-enhancing component on a surface of the gold nanoparticle to enhance a mass of the gold nanoparticle.

2. The method according to claim 1, wherein the light is ultraviolet light.

3. The method according to claim 1, wherein the metal-enhancing component is metal ions.

4. The method according to claim 3, wherein the metal ions are selected from silver (Ag) ions, copper (Cu) ions, gold (Au) ions, palladium (Pd) ions, and a combination thereof.

5. The method according to claim 1, wherein the gold nanoparticle has a diameter of about 5 nm to about 200 nm.

6. The method of claim 1, wherein the reducing is in the absence of a reducing agent.

7. A method of detecting molecular binding of a target molecule to a sensor, the method comprising:
binding a target molecule to a sensor for detecting a change in a property of a gold nanoparticle bound to the target molecule;
binding a gold nanoparticle to the target molecule bound to the sensor;
contacting the sensor with the bound gold nanoparticle and bound target molecule with a composition comprising a metal-enhancing component;
irradiating the contacted composition with a wavelength of light effective to reduce the metal-enhancing component on a surface of the gold nanoparticle to change the property of the gold nanoparticle; and
detecting the change in the property of the gold nanoparticle to detect the molecular binding of the target molecule to the sensor.

8. The method according to claim 7, wherein the change in the property of the gold nanoparticle is selected from a change in mass, optical property, and electrical property, and a combination thereof.

9. The method according to claim 7, wherein the light is ultraviolet light.

10. The method according to claim 7, wherein the metal-enhancing component is metal ions.

11. The method according to claim 10, wherein the metal ions are selected from silver (Ag) ions, copper (Cu) ions, gold (Au) ions, palladium (Pd) ions, and a combination thereof.

12. The method according to claim 7, wherein the gold nanoparticle has a diameter of about 5 nm to about 200 nm.

13. The method of claim 7, wherein the reducing is in the absence of a reducing agent.

14. The method of claim 7, wherein the target molecule is a biomolecule.

15. The method of claim 14, wherein the biomolecule is an antibody or an antigen.

16. A sensor, comprising:
a sensor having a surface to which a target molecule is bound, wherein the sensor is configured to detect a change in a property of a gold nanoparticle bound to the target molecule;
a gold nanoparticle bound to the target molecule on the surface of the sensor;
a composition comprising a metal-enhancing component, which changes a property of the gold nanoparticle upon irradiation, and which is in contact with the bound gold nanoparticle ; and
a light irradiation device which is configured to irradiate the contacted composition,
wherein the metal-enhancing component is reduced on a surface of the gold nanoparticle by light-irradiation to change the property of the gold nanoparticle, and
the sensor detects the change in the property of the gold nanoparticle.

17. The sensor according to claim 16, wherein the sensor is selected from a mass sensor, an optical sensor, an electrical sensor, and a combination thereof.

18. The sensor according to claim 16, wherein the change in the property of the metal nanoparticle is selected from a change in mass, optical property, electrical property, and a combination thereof.

19. The sensor according to claim 16, wherein the light is ultraviolet light.

20. The sensor according to claim 16, wherein the metal-enhancing component is a metal ion.

21. The sensor according to claim 20, wherein the metal ion is selected from a silver (Ag) ion, copper (Cu) ion, gold (Au) ion, palladium (Pd) ion, and a combination thereof.

22. The sensor according to claim 21, wherein the gold nanoparticle has a diameter of about 5 nm to about 200 nm.

23. The sensor of claim 16, wherein the reducing is in the absence of a reducing agent.

24. The sensor of claim 16, wherein the target molecule is a biomolecule.

25. A method of detecting molecular binding of a target antigen or a target antibody to a sensor, the method comprising:
binding the target antigen or target antibody to a surface of a sensor for detecting a change in a property of a gold nanoparticle bound to the antigen or antibody;
binding a gold nanoparticle to the antigen or antibody bound to the sensor;
contacting the sensor with the bound gold nanoparticle and bound target antigen or target antibody with a composition comprising a metal-enhancing component;
irradiating the contacted composition with a wavelength of light effective to reduce the metal-enhancing component on a surface of the gold nanoparticle to change the property of the gold nanoparticle, wherein reducing is in the absence of a reducing agent; and
detecting the change in the property of the gold nanoparticle to detect the molecular binding of the target antigen or target antibody to the sensor.

26. The method of claim 25, wherein the binding of the target antigen to the surface of the sensor and to the gold nanoparticle comprises:
binding a first antibody that specifically binds the target antigen to a surface of the sensor;
contacting the first antibody with the target antigen to specifically bind the target antigen to the first antibody; and
contacting the bound antigen with a second antibody, wherein the second antibody is bound to the gold nanop article.

27. The method of claim 25, wherein the binding of the target antibody to the surface of the sensor and to the gold nanoparticle comprises:
binding a first antigen that specifically binds the target antibody to a surface of the sensor;
contacting the first antigen with the target antibody to specifically bind the target antibody to the first antigen; and
contacting the bound target antibody with a second antigen, wherein the second antigen is bound to the gold nanop article.
